(19) United States
(12) Patent Application Publication
(10) Pub. No.: US 2013/0190725 A1
(43) Pub. Date: Jul. 25, 2013

(54) MEDICAL DEVICE HAVING TISSUE ENGAGING MEMBER AND METHOD FOR DELIVERY OF A THERAPEUTIC AGENT

(71) Applicant: ABBOTT CARDIOVASCULAR SYSTEMS INC., Santa Clara, CA (US)

(72) Inventors: Stephen D. Pacetti, San Jose, CA (US); Binh Nguyen, Newark, CA (US)

(73) Assignee: ABBOTT CARDIOVASCULAR SYSTEMS INC., Santa Clara, CA (US)

(21) Appl. No.: 13/741,099

(22) Filed: Jan. 14, 2013

Related U.S. Application Data
(63) Continuation of application No. PCT/US2011/043816, filed on Jul. 13, 2011.
(60) Provisional application No. 61/365,203, filed on Jul. 16, 2010.

(51) Int. Cl. A61M 25/10

(52) U.S. Cl. CPC A61M 25/104
USPC 604/509; 604/103.02; 606/194

(57) ABSTRACT

Medical device includes a tubular member having a proximal end and distal end defining a longitudinal axis therebetween, an expandable member proximate the distal end of the tubular member having at least one axial fold in a deflated condition, a tissue engaging member comprising at least one straight wire extending along at least part of the longitudinal axis of the expandable member, and a therapeutic agent disposed on at least the expandable member or the tissue engaging member. The at least one straight wire of the tissue engaging member is located inside the at least one fold of the expanded member when in the deflated condition. The tissue engaging member is configured for deployment at a select location upon inflation of the expandable member. A method of delivering a therapeutic agent is also provided.

### CROSS-REFERENCE TO RELATED PRIORITY

This application is a continuation of International Patent Application Serial No. PCT/US2011/043816 filed Jul. 13, 2011 and claims priority to U.S. Provisional Patent Application Ser. No. 61/365,203 entitled “Medical Device Having Tissue Engaging Member and Method for Delivery of a Therapeutic Agent” filed on Jul. 16, 2010, which are hereby incorporated by reference in their entireties herein.

### BACKGROUND OF THE DISCLOSED SUBJECT MATTER

1. Field of the Disclosed Subject Matter

The disclosed subject matter is related to the delivery of therapeutic agents from an interventional medical device. More particularly, the presently disclosed subject matter relates to an interventional device including an expandable member, such as a balloon, and a tissue engaging member for the delivery of a therapeutic agent to a vasculature.

2. Description of Related Subject Matter

Atherosclerosis is a syndrome affecting arterial blood vessels. It is characterized by a chronic inflammatory response in the walls of arteries, which is in large part due to the accumulation of lipid, macrophages, foam cells and the formation of plaque in the arterial wall. Atherosclerosis is commonly referred to as hardening of the arteries, although the pathophysiology of the disease manifests itself with several different types lesions ranging from fibrotic to lipid laden to calcific. Angioplasty is a vascular interventional technique involving mechanically widening an obstructed blood vessel, typically caused by atherosclerosis.

During angioplasty, a catheter having a folded balloon is inserted into the vasculature of the patient and is passed to the narrowed location of the blood vessel at which point the balloon is inflated to the desired size by fluid pressure. Percutaneous coronary intervention (PCI), commonly known as coronary angioplasty, is a therapeutic procedure to treat the stenotic regions in the coronary arteries of the heart, often found in coronary heart disease. In contrast, peripheral angioplasty, commonly known as percutaneous transluminal angioplasty (PTA), generally refers to the use of mechanical widening of blood vessels other than the coronary arteries. PTA is most commonly used to treat narrowing of the leg arteries, especially, the iliac, external iliac, superficial femoral and popliteal arteries. PTA can also treat narrowing of carotid and renal arteries, veins, and other blood vessels.

Although the blood vessel is often successfully widened by angioplasty, sometimes the treated region of the blood vessel undergoes vasospasm, or abrupt closure after balloon inflation or dilatation, causing the blood vessel to collapse after the balloon is deflated or shortly thereafter. One solution to such collapse is stenting the blood vessel to prevent collapse. Dissection, or perforation, of the blood vessel is another complication of angioplasty that can be improved by stenting. A stent is a device, typically a metal tube or scaffold that is inserted into the blood vessel after, or concurrently with angioplasty, to hold the blood vessel open.

While the advent of stents eliminated many of the complications of abrupt vessel closure after angioplasty procedures, within about six months of stenting a re-narrowing of the blood vessel can form, a condition known as restenosis. Restenosis was discovered to be a response to the injury of the angioplasty procedure and is characterized by a growth of smooth muscle cells and extracellular matrix—analogous to a scar forming over an injury. To address this condition, drug eluting stents were developed to reduce the reoccurrence of blood vessel narrowing after stent implantation. A drug eluting stent is a stent that has been coated with a drug, often in a polymeric carrier, that is known to interfere with the process of re-narrowing of the blood vessel (restenosis). Examples of various known drug eluting stents are disclosed in U.S. Pat. Nos. 5,649,977; 5,464,650; 5,591,227; 7,378,105; 7,445,792; and 7,335,227, each of which are hereby incorporated by reference in their entirety. However, a drawback of drug eluting stents is a condition known as late stent thrombosis. This is an event where a blood clot forms inside the stent, which can occlude blood flow.

Drug coated balloons are believed to be a viable alternative to drug eluting stents in the treatment of atherosclerotic lesions. In a study which evaluated restenosis, and the rate of major adverse cardiac events such as heart attack, bypass, repeat stenosis, or death in patients treated with drug coated balloons and drug eluting stents, the patients treated with drug coated balloons experienced only 3.7 percent restenosis and 4.8 percent MACE (material adverse coronary events) as compared to patients treated with drug eluting stents, in which restenosis was 20.8 percent and 22.0 percent MACE rate. (See, PEPCAD II study, Rotenburg, Germany).

However, drug coated balloons present certain unique challenges. For example, the drug carried by the balloon needs to remain on the balloon during delivery to the lesion site, and release from the balloon surface to the blood vessel wall when the balloon is expanded inside the blood vessel. For coronary procedures, the balloon is typically inflated for less than one minute, typically about thirty seconds. The balloon inflation time may be longer for a peripheral procedure, however typically even for peripheral procedures the balloon is expanded for less than 5 minutes. Due to the short duration of contact between the drug coated balloon surface and the blood vessel wall, the balloon coating must exhibit efficient therapeutic agent transfer and/or efficient drug release during inflation. Thus, there are challenges specific to drug delivery via a drug coated or drug eluting balloon that are not present with a drug eluting stent.

Thus there remains a need, and an aim of the disclosed subject matter is directed towards, a medical device and method for increasing the delivery of a therapeutic agent to a vasculature. Furthermore, there remains a need for a more controlled angioplasty procedure.

### SUMMARY OF THE DISCLOSED SUBJECT MATTER

The purpose and advantages of the disclosed subject matter will be set forth in and are apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

To achieve these and other advantages and in accordance with the purpose of the disclosed subject matter, as embodied and broadly described, the disclosed subject matter includes a medical device. The medical device includes a tubular member having a proximal end and distal end defining a longitudinal axis therebetween, an expandable member proximate the distal end of the tubular member having at least one axial fold in a deflated condition, a tissue engaging member comprising at least one straight wire extending along at least part of the longitudinal axis of the expandable member, and a therapeutic agent disposed on at least the expandable member or the tissue engaging member. The at least one straight wire of the tissue engaging member is located inside the at least one fold of the expanded member when in the deflated condition. The tissue engaging member is configured for deployment at a select location upon inflation of the expandable member.

The disclosed subject matter also includes a method of delivering a therapeutic agent. The method includes delivering at least a portion of a medical device within a vasculature. The medical device includes a tubular member having a proximal end and distal end defining a longitudinal axis therebetween, an expandable member proximate the distal end of the tubular member and having at least one axial fold, a tissue engaging member comprising at least one straight wire extending along at least part of a longitudinal axis of the expandable member and located inside the at least one axial fold of the expanded member, and a therapeutic agent disposed on at least the expandable member or the tissue engaging member. The method further includes inflating the expandable member to deploy the tissue engaging member at a select location and to engage the therapeutic agent with a vessel wall, deflating the expandable member, and withdrawing the medical device from the vasculature. The method and medical device can include any number of the features described in greater detail below.

Further in accordance with the disclosed subject matter, an alternative medical device is provided. The medical device includes a tubular member having a proximal end and distal end defining a longitudinal axis therebetween, an expandable member proximate the distal end of the tubular member having at least one axial fold in a deflated condition, and a tissue engaging member comprising at least one straight wire extending along at least part of the longitudinal axis of the expandable member. The at least one straight wire of the tissue engaging member is located inside the at least one fold of the expanded member when in the deflated condition. The tissue engaging member is configured for deployment at a select location upon inflation of the expandable member.

The disclosed subject matter also includes a method of treating a vasculature. The method includes delivering at least a portion of a medical device within a vasculature. The medical device includes a tubular member having a proximal end and distal end defining a longitudinal axis therebetween, an expandable member proximate the distal end of the tubular member and having at least one axial fold, and a tissue engaging member comprising at least one straight wire extending along at least part of a longitudinal axis of the expandable member and located inside the at least one axial fold of the expanded member. The method further includes inflating the expandable member to deploy the tissue engaging member at a select location and to engage the expandable member with a vessel wall, deflating the expandable member, and withdrawing the medical device from the vasculature. The method and medical device can include any number of the features described in greater detail below.

It is to be understood that both the foregoing general description and the following detailed description are exemplary and are intended to provide further explanation of the disclosed subject matter claimed.

The accompanying drawings, which are incorporated in and constitute part of this specification, are included to illustrate and provide a further understanding of the method and system of the disclosed subject matter. Together with the description, the drawings serve to explain the principles of the disclosed subject matter.

### BRIEF DESCRIPTION OF THE DRAWINGS

FIG. 1 is a schematic side view with partial cross-section of a representative balloon catheter in accordance with the disclosed subject matter.

FIG. 1A is a cross-sectional view taken along lines A-A in FIG. 1.

FIG. 1B is a cross-sectional view taken along lines B-B in FIG. 1.

FIG. 2 is a cross sectional view of an expandable member and tissue engaging member in accordance with the disclosed subject matter.

FIGS. 3A, 3B, and 3C are cross sectional views of the embodiment of FIG. 2 showing the expandable member being inflated.

FIG. 4 is a schematic side view of an expandable member and tissue engaging member in accordance with the disclosed subject matter.

FIG. 5 is a cross sectional view of an expandable member and tissue engaging member in accordance with the disclosed subject matter.

FIG. 6 is a schematic side view of a portion of tubular member and collar in accordance with the disclosed subject matter.

FIG. 7 is a schematic side view of the expandable member, tissue engaging member, and collar in accordance with the disclosed subject matter.

FIG. 8 is a schematic side view of an expandable member and tissue engaging member in accordance with the disclosed subject matter.

FIG. 9 is a schematic side view of the embodiment of FIG. 8 in the inflation condition.

FIG. 10 is a schematic side view of an expandable member and tissue engaging member in accordance with the disclosed subject matter.

### DETAILED DESCRIPTION OF THE PREFERRED EMBODIMENT

Reference will now be made in detail to the preferred embodiments of the disclosed subject matter, an example of which is illustrated in the accompanying drawing. The method and corresponding steps of the disclosed subject matter will be described in conjunction with the detailed description of the system.

As disclosed herein, the devices and methods presented can be used for treating the lumen of a patient. In particular, the disclosed subject matter is particularly suited for treatment of the cardiovascular system of a patient, such as performance of angioplasty and delivery of a therapeutic agent to a vasculature.

In accordance with the disclosed subject matter, a medical device includes a tubular member having a proximal end and distal end defining a longitudinal axis therebetween, an expandable member proximate the distal end of the tubular member having at least one axial fold in a deflated condition, a tissue engaging member comprising at least one straight wire extending along at least part of the longitudinal axis of the expandable member, and a therapeutic agent disposed on at least the expandable member or the tissue engaging member. The at least one straight wire of the tissue engaging member is located inside the at least one fold of the expanded member when in the deflated condition. The tissue engaging member is configured for deployment at a select location upon inflation of the expandable member.

The disclosed subject matter also includes a method of delivering a therapeutic agent including delivering at least a portion of a medical device within a vasculature. The medical device includes a tubular member having a proximal end and distal end defining a longitudinal axis therebetween, an expandable member proximate the distal end of the tubular member and having at least one axial fold, a tissue engaging member comprising at least one straight wire extending along at least part of a longitudinal axis of the expandable member and located inside the at least one axial fold of the expanded member, and a therapeutic agent disposed on at least the expandable member or the tissue engaging member. The method further includes inflating the expandable member to deploy the tissue engaging member at a select location and to engage the therapeutic agent with a vessel wall, deflating the expandable member, and withdrawing the medical device from the vasculature. The medical device will be described in conjunction with the method for purpose of understanding.

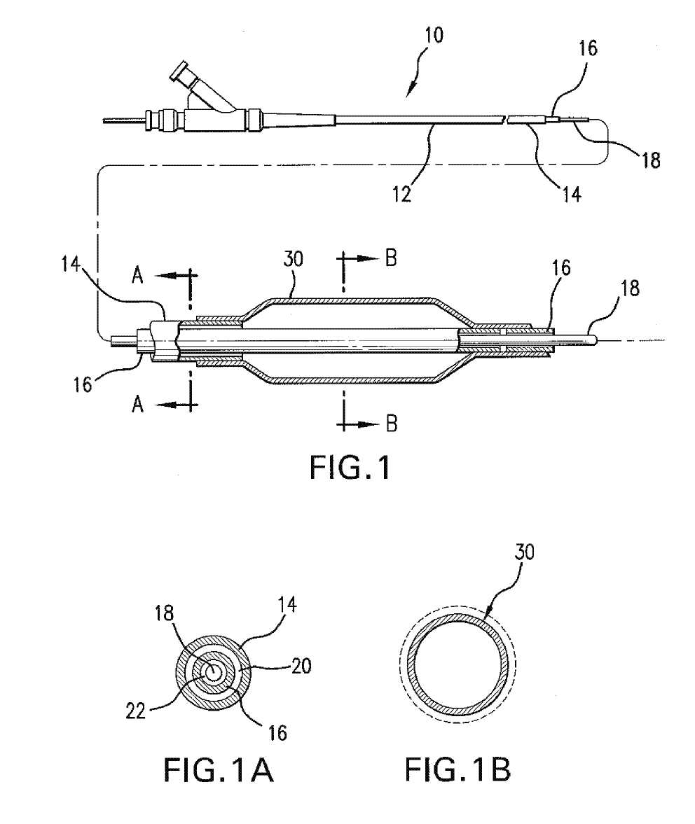

For purpose of explanation and illustration, and not limitation, an exemplary embodiment of a medical device, at least a portion of which is delivered within a vasculature, is shown schematically in FIG. 1. Particularly, and as illustrated, the medical device embodied herein can be a balloon catheter 10, which includes an tubular member or elongated catheter shaft 12 having a proximal end and distal end defining a longitudinal axis therebetween and an expandable member 30 located proximate the distal end of the catheter shaft. The expandable member, or balloon as depicted herein, has an outer surface and an inner surface disposed at the distal end portion of the catheter shaft.

The elongated catheter shaft 12 comprises an outer tubular member 14 and an inner tubular member 16. The outer tubular member 14 defines an inflation lumen 20 disposed between the proximal end portion and the distal end portion of the catheter shaft 12. Specifically, as illustrated in FIG. 1A, the coaxial relationship of this representative embodiment defines an annular inflation lumen 20 between the inner tubular member 16 and the outer tubular member 14. The expandable member 30 is in fluid communication with the inflation lumen 20. The inflation lumen can supply an inflation medium under positive pressure and can withdraw the inflation medium, i.e. provide negative pressure, from the expandable member. The expandable member 30 can thus be inflated and deflated. The elongated catheter is sized and configured for delivery within a vasculature and particularly through a tortuous anatomy, and can further include a guidewire lumen 22 that permits it to be delivered over a guidewire 18. As illustrated in FIG. 1A, the inner tubular member 16 defines the guidewire lumen 22 for the guidewire 18. Although FIGS. 1 and 1b illustrate the guidewire lumen as having an over-the-wire (OTW) construction, the guidewire lumen can be configured as a rapid-exchange (RX) construction, as is well known in the art.

A wide variety of expandable members 30, such as balloons, and constructs are known and suitable for use in accordance with the disclosed subject matter. For example, the expandable member can be made from polymeric material such as compliant, non-compliant or semi-compliant polymeric material or polymeric blends.

In one embodiment, the polymeric material is compliant such as, but not limited to, a polyamide/polyether block copolymer (commonly referred to as PEBA or polyether-block-amide). Preferably, the polyamide and polyether segments of the block copolymers can be linked through amide or ester linkages. The polyamide block can be selected from various aliphatic or aromatic polyamides known in the art. Preferably, the polyamide is aliphatic. Some non-limiting examples include nylon 12, nylon 11, nylon 9, nylon 6, nylon 6/12, nylon 6/11, nylon 6/9, and nylon 6/6. Preferably, the polyamide is nylon 12. The polyether block can be selected from various polyethers known in the art. Some non-limiting examples of polyether segments include poly(tetramethylene ether), tetramethylene ether, polyethylene glycol, polypropylene glycol, poly(pentamethylene ether) and poly(hexamethylene ether). Commercially available PEBA material can also be utilized such as for example, PEBAX® materials supplied by Arkema (France). Various techniques for forming a balloon from polyamide/polyether block copolymer are known in the art. One such example is disclosed in U.S. Pat. No. 6,406,457 to Wang, the disclosure of which is incorporated by reference in its entirety.

In another embodiment, the balloon material is formed from polyamides. Preferably, the polyamide has substantial tensile strength, is resistant to pin-holing even after folding and unfolding, and is generally scratch resistant, such as those disclosed in U.S. Pat. No. 6,500,148 to Pinchuk, the disclosure of which is incorporated herein by reference in its entirety. Some non-limiting examples of polyamide materials suitable for the balloon include nylon 12, nylon 11, nylon 9, nylon 69 and nylon 66. Preferably, the polyamide is nylon 12. Other suitable materials for constructing non-compliant balloons are polyesters such as polyethylene terephthalate) (PET), Hytrel thermoplastic polyester, and poly(ethylene.

In another embodiment, the balloon is formed of a polyurethane material, such as TECOTHANE® (Thermedics). TECOTHANE® is a thermoplastic, aromatic, polyether polyurethane synthesized from methylene disocyanate (MDI), polytetramethylene ether glycol (PTMEG) and 1,4 butanediol chain extender. TECOTHANE® grade 1065D is presently preferred, and has a Shore durometer of 65D, an elongation at break of about 300%, and a high tensile strength at yield of about 10,000 psi. However, other suitable grades can be used, including TECOTHANE® 1075D, having a Shore D hardness of 75. Other suitable compliant polymeric materials include ENGAGE® (DuPont Dow Elastomers (an ethylene alpha-olefin polymer)) and EXACT® (Exxon Chemical), both of which are thermoplastic polymers. Other suitable compliant materials include, but are not limited to, elastomeric silicones, latexes, and urethanes.

The compliant material can be cross linked or uncrosslinked, depending upon the balloon material and characteristics required for a particular application. The presently preferred polyurethane balloon materials are not crosslinked. However, other suitable materials, such as the polyolefinic polymers ENGAGE® and EXACT®, are preferably crosslinked. By crosslinking the balloon compliant material, the final inflated balloon size can be controlled. Conventional crosslinking techniques can be used including thermal treatment and E-beam exposure. After crosslinking, initial pressurization, expansion, and preshrinking, the balloon will thereafter expand in a controlled manner to a reproducible diameter in response to a given inflation pressure, and thereby avoid overexpanding the balloon to an undesirably large diameter.

In one embodiment, the balloon is formed from a low tensile set polymer such as a silicone-polyurethane copolymer. Preferably, the silicone-polyurethane is an ether urethane and more specifically an aliphatic ether urethane such as PURSIL AL 575A and PURSIL AL10, (Polymer Technology Group), and ELAST-EON 3-70A (Elastomedics), which are silicone polyether urethane copolymers, and more specifically, aliphatic ether urethane cosiloxanes. In an alternative embodiment, the low tensile set polymer is a diene polymer. A variety of suitable diene polymers can be used such as, but not limited to, an isoprene such as an AB and ABA polystyrene-block-isoprene), a neoprene, an AB and ABA poly(styrene-block-butadiene) such as styrene butadiene styrene (SBS) and styrene butadiene rubber (SBR), and 1,4-polybutadiene. Preferably, the diene polymer is an isoprene including isoprene copolymers and isoprene block copolymers such as poly(styrene-block-isoprene). A presently preferred isoprene is a styrene-isoprene-styrene block copolymer, such as Kraton 1161K available from Kraton, Inc. However, a variety of suitable isoprenes can be used including HT 200 available from Apex Medical, Kraton R 310 available from Kraton, and isoprene (i.e., 2-methyl-1,3-butadiene) available from Dupont Elastomers. Neoprene grades useful in the disclosed subject matter include HT 501 available from Apex Medical, and neoprene (i.e., polychloroprene) available from Dupont Elastomers, including Neoprene G, W, T and A types available from Dupont Elastomers. Examples of other balloon and catheter embodiments which can be employed in accordance with the disclosed subject matter include U.S. Pat. Nos. 4,748,982; 5,496,346; 5,626,600; 5,300,085; and 6,406,457 and application Ser. Nos. 12/371,426; 11/539,944; and 12/371,422, each of which is hereby incorporated by reference in its entirety.

In accordance with another aspect of the disclosed subject matter, the outer surface of the balloon can be modified. In this regard, the balloon surface can include a textured surface, roughened surface, voids, spines, channels, dimples, pores, or microcapsules or a combination thereof.

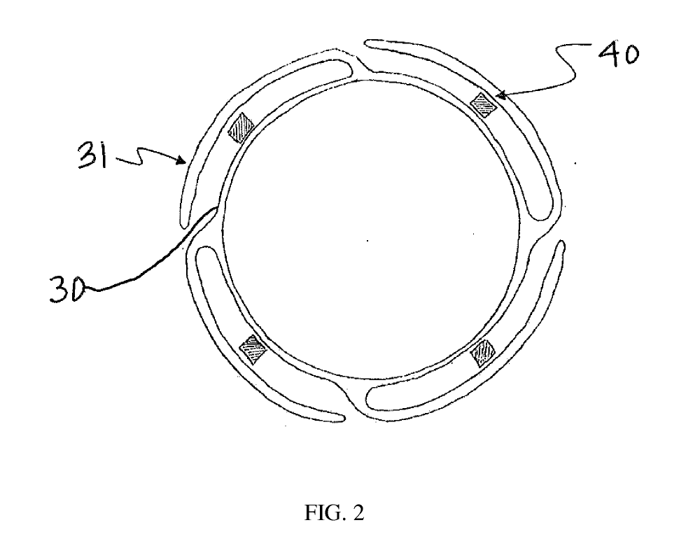

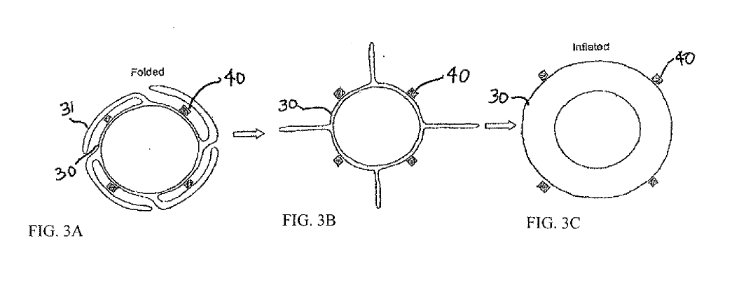

In accordance with the disclosed subject matter, the expandable member of the medical device can have at least one fold defined therein. For purpose of explanation and illustration, and not limitation, an exemplary embodiment of an expandable member is shown schematically in FIG. 2. As shown in FIG. 2, the expandable member 30 preferably has at least one axial fold 31 in a deflated condition. The axial fold 31 lies straight along the length of the expandable member. The expandable member is configured so as to have a folded configuration and a fully expanded configuration, as shown in FIGS. 3A-3C, for the purpose of illustration and not limitation. Generally, the formation of folds can be performed using heat and pressure to farm or define creases in the material of the balloon. Examples of folded balloons are disclosed, for purpose of illustration in U.S. Pat. Nos. 6,494,906; 6,478,807; and 5,911,452, each of which is hereby incorporated by reference in its entirety.

As will be described below in more detail, a therapeutic agent can be disposed on at least the expandable member or the tissue engaging member, or both. During delivery of the medical device to the target site, and subsequent to inflation of the expandable member, it is desired to keep the therapeutic agent in place. During inflation, relative motion between the expandable member and the tissue engaging member can result in therapeutic agent being released from the balloon. Locating the tissue engaging member within the balloon folds can reduce this relative motion. Particularly, locating the tissue engaging member at certain locations within the folds can reduce the relative motion between the expandable member and tissue engaging member, one such location being in the middle of the balloon folds, as depicted in FIG. 3A.

The folds also protect the tissue engaging member and the coating of therapeutic agent (described below in more detail) during delivery of the expandable member through the body lumen to the target site, such that drug loss and injury to the vessel are minimized. The folds can be utilized to protect the coating containing therapeutic agent from releasing from at least a portion of the expandable member during the movement of the medical device through the body lumen. The folds also protect the vessel wall during movement of the medical device through the body lumen. Furthermore, the folds can also protect the coating during shipping and storage before use.

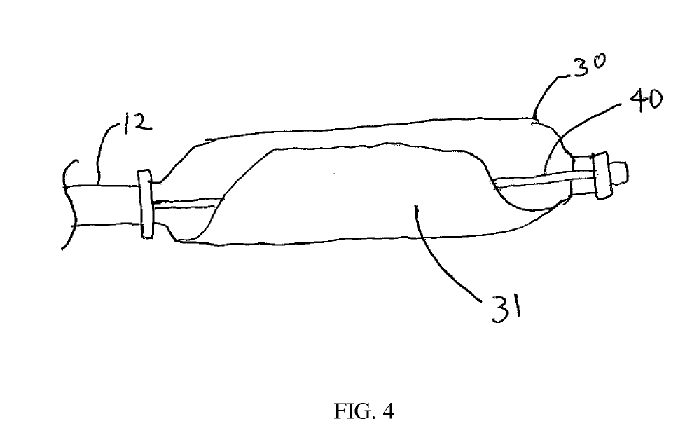

For purpose of explanation and illustration, and not limitation, an exemplary embodiment of the expandable member and a tissue engaging member is shown schematically in FIG. 4. The tissue engaging member 40 is located proximate the expandable member 30. The tissue engaging member has a collapsed configuration for delivery and an expanded configuration for engagement with the vessel wall. As shown is FIGS. 2-4, the tissue engaging member includes at least one straight wire extending along at least part of the longitudinal axis of the expandable member. Preferably, the at least one straight wire of the tissue engaging member 40 is located inside the at least one fold 31 of the expanded member 30 when in the deflated condition as shown in FIG. 4. Furthermore, the tissue engaging member is configured for deployment at a select location upon inflation of the expandable member. For example, the tissue engaging member is an expandable member and can be shape-set or thermally trained to be in the collapsed state, such that it is expanded by inflation of the expandable member. In such an embodiment, upon deflation, the tissue engaging member will return to the smaller collapsed profile. In a preferred embodiment, this may be accomplished by using nitinol in a super elastic state with the shape memory set to the collapsed state. Otherwise, a number of elastic or spring like alloys/metals may be used, such as Elgiloy. Alternatively, and if desired, the tissue engaging member can be a self-expanding structure, which is held in a collapsed position by the folds in the expandable member. If the self-expanding forces are low enough by use of small cross section tissue engaging members, and the folds fowled tightly enough, then the balloon folds themselves may hold the tissue engaging member in a collapsed state. In this embodiment, the medical device includes a suitable mechanism to collapse the tissue engaging member after deflation, but before withdrawing the medical device form the vasculature. One design can include connections, hooks, loops, or bonding to connect the tissue engaging member to the expandable member. When a vacuum is applied to the expandable member, its collapse will also cause a reduction in the diameter of the engaging member. In another embodiment, a sheath can be present on the medical device. Such a sheath can be present during delivery of the device to the target site, or the sheath may only be used to facilitate collapse of the expanding member.

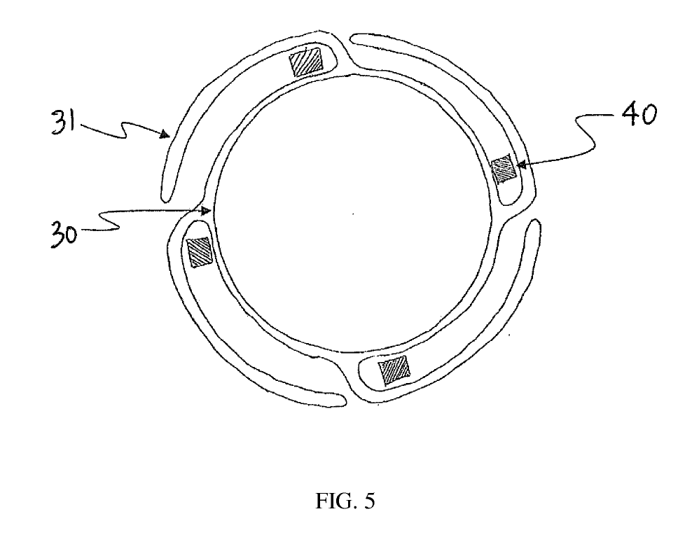

The tissue engaging member can have any suitable configuration. For example, the tissue engaging member can include a plurality of continuous longitudinal wires. Preferably, the tissue engaging member includes at least one straight wire extending along at least part of the longitudinal axis of the expandable member. In a preferred embodiment, the expandable member can include a plurality of folds in the deflated condition with at least one straight wire located inside each fold. By positioning the wires inside the folds, the relative motion between the wire and the surface of the expandable member is reduced. Furthermore, one straight wire of the tissue engaging member can be located in substantially the middle of each fold of the expandable member when in the deflated condition, as shown in FIG. 2-4, for the purpose of illustration. Positioning the wires in the middle of the folds reduces the relative motion between the scoring wire and the surface of the expandable member during inflation to near zero, as is demonstrated for the purpose of illustration in FIGS. 3A-C. Alternatively, the wires can be located at other positions in the folds or even located outside the folds. For example, the wires can be located proximate where the fold connects to the body, as shown in FIG. 5 for the purpose of illustration and not limitation.

In accordance with another aspect of the disclosed subject matter, the tissue engaging member has a tissue engaging member to artery ratio, which represents the percent of the luminal area occupied by the tissue engaging member when expanded against the luminal vessel wall, between about 1 to about 50% and preferably between about 2.5 to about 25%.

In accordance with another aspect of the disclosed subject matter, the tissue engaging member can be fixed at least one of either the proximal or distal end. For example, the distal end of the at least one straight wire of the tissue engaging member can be fixedly attached proximate to a distal end of the expandable member. The tissue engaging member, or each individual wire or element, can be joined to the tubular member by a number of known means, such as adhesively bonded, thermo bonded, welded, crimped, etc. For example, the tissue engaging member can include a collar encircling and/or joined to the tubular member. Additionally or alternatively, the wires of the tissue engaging member can be slipped or mounted into small formations on the tubular member or terminate into a collar that is affixed to the tubular member.

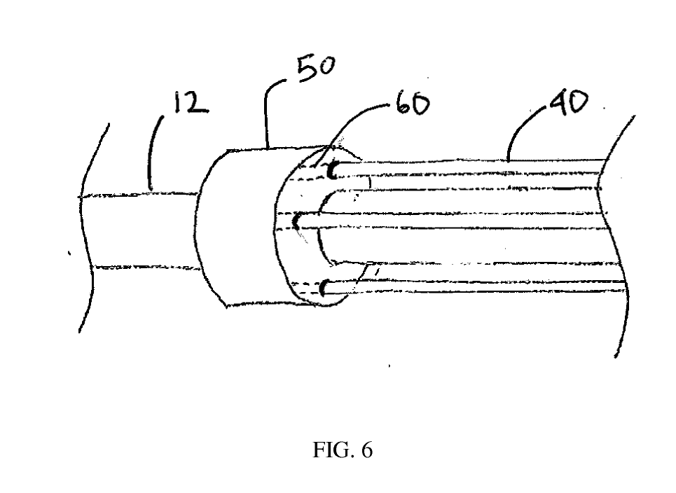

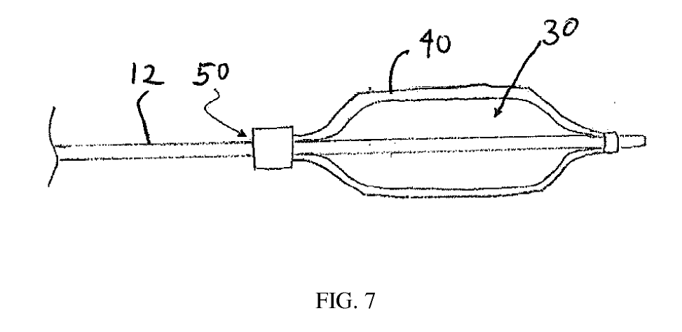

Additionally or alternatively, the distal or proximal end, or both, of the at least one straight wire of the tissue engaging member can be attached to a slidable collar disposed on the tubular member. As shown in FIGS. 6 and 7 for the purpose of illustration and not limitation, the proximal end of the at least one straight wire of the tissue engaging member is attached to a slidable collar 50 disposed on the tubular member 12 proximate the proximal end of the expandable member 30. The collar 50 encircles the tubular member 12, but is not fixedly attached to the tubular member so that it can float or slide on the tubular member when the tissue engaging member is expanded and collapsed. When the expandable member is inflated, the proximal collar will move distally towards the expandable member as the tissue engaging member expands to take the shape of the expandable member. Therefore, the length of the wires and the initial location of the proximate collar must be designed to accommodate the maximum outer diameter of the expandable member in the inflated condition. After treatment is complete and the expandable member is deflated, the proximal collar will slide proximally returning to about its original position in the deflated condition.

The collar 50 can be made of a polymeric extrusion having at least one sub-lumen 60 in the wall of the extrusion, as best shown in FIG. 6. For example, the collar can be made of a polymeric multi-lumen extrusion where there are sub-lumens in the wall of the primary tubing extrusion. The collar can be made of any other suitable material, including but not limited to nitinol. The proximal end of the at least one straight wire 40 of the tissue engaging member can be secured in a corresponding sub-lumen 60 of the collar, for example using an adhesive or any other suitable connecting means. In one embodiment, the wires of the tissue engaging member and the collar, whether slidable or fixedly attached, are all made from a single piece by laser cutting and electropolishing a tube, for example made of nitinol.

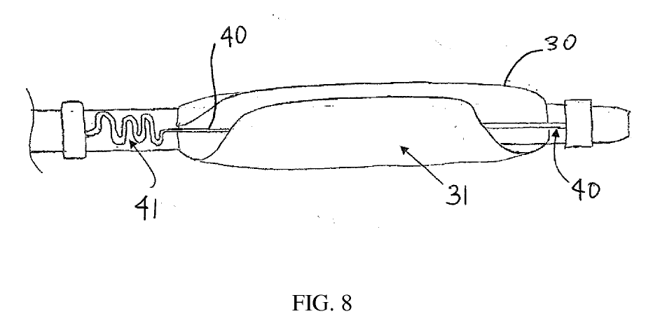

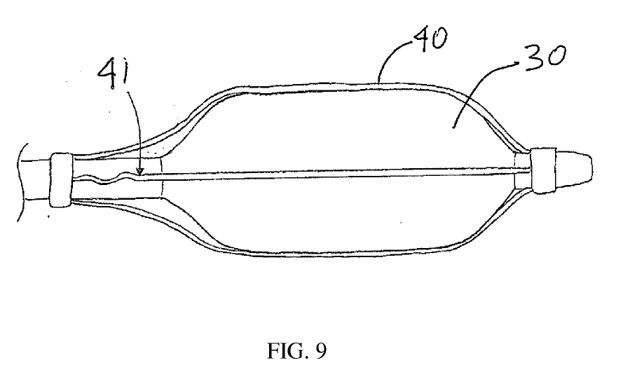

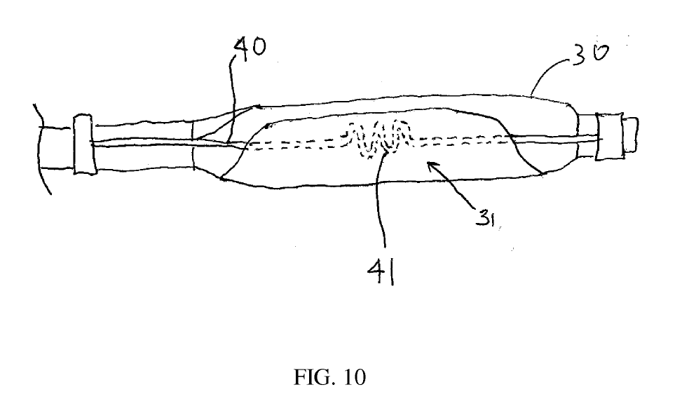

If the tissue engaging member is fixedly attached at both ends, then the tissue engaging member can further include a non-linear portion to allow for the wires to expand upon inflation of the expandable member. For example and as is shown in FIG. 8 for the purpose of illustration and not limitation, the tissue engaging member 40 can further include a non-linear portion 41 at the proximal end of the least one straight wire, and the non-linear portion 41 has a proximal end fixedly attached to the tubular member 12 proximate the proximal end of the expandable member 30. For the purpose of illustration and not limitation, FIG. 9 demonstrates how the embodiment of FIG. 8 would appear after inflation. As shown in FIG. 9, the non-linear portions at least somewhat straighten when the tissue engaging member is expanded upon inflation of the expandable member. Alternatively or additionally, and as shown in FIG. 10 for the purpose of illustration and not limitation, the tissue engaging member can further include a non-linear portion adjacent to the straight wire of the tissue engaging member and located inside a fold 31 of the expanded member 30 when in the deflated condition. Additionally or alternatively, the wire can include a 2-dimensional array or pattern of wires adjacent to the straight wire of the tissue engaging member and located inside a fold 31 of the expanded member 30 when in the deflated condition. Such an arrangement can provide a more uniform injury to the vessel wall.

In accordance with one aspect of the disclosed subject matter, at least a portion of the at least one straight wire of the tissue engaging member can be secured to a surface of the expandable member. This configuration can assure that the wires of the tissue engaging member stay in their relative positions during inflation to provide a more uniform injury, controlled angioplasty procedure, and reduced loss of therapeutic agent during inflation. Furthermore, having at least a portion of the wire secured to the balloon assists the tissue engaging member in collapsing upon deflation of the expandable member. The tissue engaging member can be coupled to the balloon using a variety of known techniques such as, but not limited to, using solvents or adhesives, or by formations provided on the surface of the expandable member to capture or engage the wires or elements.

The tissue engaging member can be made of a variety of suitable materials. For example, the tissue engaging member can be metallic, a polymer, an elastomer, or a metallic alloy. Non-limiting examples of suitable materials include nitinol, elgiloy, stainless steel, cobalt-chromium, alloys thereof, and combinations thereof. In the case of cobalt-chromium alloys and stainless steel alloys, it is preferred to work harden the materials to provide the desired elasticity for expansion. Suitable polymers include polyethylene, polypropylene, poly(ethylene terephthalate), Dytrel, polyurethane, nylon-6, nylon-66, nylon-12, PEBAX, poly(vinylidene fluoride), poly(tetrafluoroethylene), or poly(vinylidene fluoride-co-hexafluoropropylene). If a metallic material, a polymer, or other suitable material is used, the tissue engaging member can be laser cut from a single tube. In one embodiment, the tissue engaging member can be laser cut at the fully expanded size and then fused to the expandable member, for example by placing the tissue engaging member in a constraining tube, inflating the expandable member inside the balloon, and then heating the tube to fuse the tissue engaging member to the expandable member.

Each element or wire of the tissue engaging member can have any suitable dimensions, for example from about 0.05 microns to about 250 microns in diameter, width, and/or height. The elements or wires of the tissue engaging member can have a cross sectional configuration of a variety of shapes and ratios of width to height depending upon desired performance characteristics. Non-limiting examples of suitable cross section configurations include circular, triangular, rectangular, square, or other polygonal cross section configurations. Using a tissue engaging member in accordance with the disclosed subject matter allows for more design freedom and the ability to make wires of an optimal configuration when compared to a conventional stents because the expandable member does not remain in the body after the medical procedure is complete.

The tissue engaging member can include a coating disposed on the outer surface thereof. The coating can include a therapeutic agent, among other components, as described below or more detail.

In accordance with another aspect of the disclosed subject matter, the tissue engaging member can include protrusions or other raised surfaces configured to contact or penetrate the arterial wall of a vessel, which can increase the uptake of the therapeutic agent and provide a more uniform injury to the vessel wall. A coating containing therapeutic agent, and/or other components as described in more detail below, can be disposed on the protrusions such that when expanded, the coating and/or therapeutic agent coats the tissue of the arterial wall. Additionally or alternatively, the surface of the tissue engaging member can be roughened to provide better penetration into the wall of the vessel to enhance drug transfer.

In accordance with another aspect of the disclosed subject matter, a therapeutic agent is disposed on at least the expandable member or the tissue engaging member, or both. The therapeutic agent can be for the treatment of a disease. Examples of suitable therapeutic agents include anti-proliferative, anti-inflammatory, antineoplastic, antiplatelet, anti-coagulant, anti-fibrin, antithrombotic, antimitotic, antibiotic, antiallergic and antioxidant compounds. Such therapeutic agents can be, again without limitation, a synthetic inorganic or organic compound, a protein, a peptide, a polysaccharides and other sugars, a lipid, DNA and RNA nucleic acid sequences, an antisense oligonucleotide, an antibodies, a receptor ligands, an enzyme, an adhesion peptide, a blood clot agent including streptokinase and tissue plasminogen activator, an antigen, a hormone, a growth factor, a ribozyme, and a retroviral vector.

Preferably, however, the therapeutic agents include a cytostatic drug. The term “cytostatic” as used herein means a drug that mitigates cell proliferation but allows cell migration. These cytostatic drugs, include for the purpose of illustration and without limitation, macrolide antibiotics, rapamycin, everolimus, zotaroliumus, biolimus, temsirolimus, deforolimus, novolimus, myolimus, structural derivatives and functional analogues of rapamycin, structural derivatives and functional analogues of everolimus, structural derivatives and functional analogues of zotarolimus and any macrolide immunosuppressive drugs. The term “cytotoxic” as used herein means a drug used to inhibit cell growth, such as chemotherapeutic drugs. Some non-limiting examples of cytotoxic drugs include vincristine, actinomycin, cisplatin, taxanes, paclitaxel, and protaxel. Other preferred drugs include dexamethasone, statins, sirolimus, and tacrolimus.

In addition to the therapeutic agent, any of a variety of fluid compositions can be applied to the expandable member or the tissue engaging member, or both. The fluid can include compounds or additives, such as polymers, binding agents, plasticizers, solvents, surfactants, additives, chelators, fillers, excipients, and the like, or combinations thereof. Suitable excipients, binding agents and other components include those described in detail in U.S. patent application Ser. No. 12/636,079, which is hereby incorporated by reference in its entirety. Preferred excipients include poly(ethylene glycol) (PEG), polyvinylpyrrolidone (PVP), polyoxyethylene sorbitan monooleate (tweens), poloxamer triblock copolymers of poly(ethylene oxide)-poly(propylene oxide)-poly(ethylene oxide) (Pluronics), carboxymethyl cellulose (CMC), and PEG phospholipids such as 1,2-distearolyl-sn-glycero-3-phosphoethanolamine-N-(methoxy(polyethylene glycol)-2000) (PEG-PE). Preferred plasticizers include PEG, propylene glycol, N-methylpyrrolidone (NMP), glycerin, and tweens. Examples of possible compounds include zotarolimus, PVP and glycerol. In one embodiment the therapeutic agent can be provided in liquid form or dissolved in a suitable solvent. In another embodiment, the therapeutic agent is provided as a particulate and mixed in a suitable carrier for application as a fluid.

The fluid compositions, such as the therapeutic agents, can be applied to the expandable member or the tissue engaging member using a variety of know techniques, such as spraying (air-atomization, ultrasonic, electrostatic, piezoelectric, etc.), spray drying, pneumatic spray, spray with patterning, electrospinning, direct fluid application, dip-coating, spin-coating, pipette coating, syringe coating, vapor deposition, roll coating, micro-droplet coating, ultrasonic atomization, or other means as known to those skilled in the art. The coating can be applied over at least a length or the entirety of the expandable member. By way of example, and not limitation, certain coating processes that can be used with the instant disclosed subject matter are described in U.S. Pat. No. 6,669,980 to Hansen; U.S. Pat. No. 7,241,344 to Worsham; U.S. Publication No. 2004/0234748 to Stenzel; and U.S. Patent Application Ser. No. 61/345,575, the entire disclosures of which are hereby incorporated by reference. In accordance with one embodiment of the disclosed subject matter, the coating can be applied to either a folded or inflated balloon. Furthermore, the coating can be directly applied into the folds of the folded balloons. The coating characteristics are affected by process variables. For example, for dip-coating process, coating quality and thickness can vary as an effect of variables such as number, rate, and depth of dips along with drying time and temperature.

In accordance with another aspect of the disclosed subject matter, the expandable member or tissue engaging member can include microcapsules on its outer surface. In this regard, the microcapsules are configured to encompass the coating and/or therapeutic agent. Upon inflation of the expandable member the microcapsules located on the surface of the expandable member contact the tissue of the arterial wall. Alternatively, the microcapsules can be formed in the wall of the expandable member surface or on the tissue engaging member. The coating and/or therapeutic agent can be released from the microcapsules by fracturing of the microcapsules and/or diffusion from the microcapsule into the arterial wall. The microcapsules can be fabricated in accordance with the methods disclosed in U.S. Pat. No. 5,1023,402 to Dror or U.S. Pat. No. 6,129,705 to Grantz and the patents referenced therein, each of which is incorporated herein by reference in its entirety.

During delivery and before inflation, the expanded member can be under negative pressure to help keep the expandable member and the tissue engaging member in a collapsed state. After delivering the portion of the medical device including the expandable member and tissue engaging member to a select location within the vasculature, the expandable member is inflated to deploy the tissue engaging member at a select location and to engage the therapeutic agent with a vessel wall. Any techniques known in the art for inflating the expandable member can be used. For example, if the expandable member is a balloon, an inflation lumen located within the tubular member can supply an inflation medium under positive pressure to the expandable member, thus causing the expandable member to inflate. The expandable member is inflated at least until the tissue engaging member contacts the vessel wall. Preferably, the expandable member can be inflated to a diameter about equal to the diameter of a reference vessel or up to about 30% larger that the diameter of the reference vessel. The expandable member can be inflated for about 5 minutes or less, depending on the treatment performed and the location of the lumen in the boy. If desired, the tissue engaging member can be rotated, for example to cause denudation and vessel injury if that is the intent such as with a preclinical animal model.

Inflating the expandable member will cause the expandable member to contact the vessel wall and the therapeutic agent will be rapidly released. Furthermore, inflating the expandable member will engage the tissue engaging member with the vessel wall. The inflation can urge the tissue engaging structure into the tissue of the vessel wall to assist in increasing the therapeutic agent transfer. The tissue engaging member preferably is designed to transmit force evenly about the circumference of the vessel wall, thus causing controlled injury to the vasculature, which increases the efficiency the transfer of therapeutic agent to the body lumen. Thus, the method described herein can provide a controlled angioplasty treatment to the vessel wall and drug delivery and transfer to the vessel wall in one step. However, if desired, a predilation step can be performed. Furthermore, the expandable member can undergo multiple inflations, and/or the device can be rotated during or between inflations.

After the expandable member has been inflated for a sufficient time to widen the obstructed vessel wall and/or to transfer the therapeutic drug to the vessel wall, the expandable member is deflated. Preferably, the expandable member refolds during deflation to return to about its original folded configuration covering the wires of the tissue expanding member. A variety of techniques known in the art for deflating the expandable member can be used. For example, an inflation lumen located within the tubular member can withdraw the inflation medium, i.e. provide negative pressure, from the expandable member thus causing the expandable member to deflate. A number of known devices and techniques can be used for withdrawing desired amounts of inflation medium. For example, a deflation device such as a syringe pump, having a gas-tight syringe can be attached to the inflation lumen of the expandable member. The deflation device allows for automated, repeatable, and controlled amount of fluid withdrawn by volume from the expandable member. This is advantageous since it reduces or eliminates the variability inherent in a human operator controlled method or apparatus. Alternative devices include an indeflator or vacuum box to draw a vacuum on the expandable member. The indeflator or vacuum box is placed in fluid communication with the inflation lumen of the expandable member to remove the fluid located in the expandable member.

After deflating the expandable member, the medical device is withdrawn from the vasculature. Preferably, the tissue engaging member will collapse before the medical device is withdrawn from the vasculature. In accordance with one aspect of the disclosed subject matter, if the tissue engaging member is self-expandable, the medical device can include connections, hooks, loops, bonding or any other suitable configuration to collapse tissue engaging member after deflation but before withdrawing the medical device from the vasculature.

While the disclosed subject matter is described above in connection with the delivery of a therapeutic agent to a vasculature, the devices and methods described herein can be used without a therapeutic agent. For example, the methods and devices described herein can be used in angioplasty procedures without drug delivery. Use of an expandable member having a tissue engaging member as described herein provides a controlled angioplasty procedure and improved vascular response to reduce the occurrence of negative side effects (dissections, focal vessel damage, stenosis, and restenosis). For example, the wires anchoring the balloon in place during the angioplasty procedure can distribute the force of the balloon in a controlled manner, thus reducing trauma and increasing uniformity of injury to the vasculature.

Further in accordance with the disclosed subject matter, an alternative medical device and a method of treating a vasculature is provided. The method includes delivering at least a portion of a medical device within a vasculature. The medical device includes a tubular member having a proximal end and distal end defining a longitudinal axis therebetween, an expandable member proximate the distal end of the tubular member and having at least one axial fold, and a tissue engaging member comprising at least one straight wire extending along at least part of a longitudinal axis of the expandable member and located inside the at least one axial fold of the expanded member. The method further includes inflating the expandable member to deploy the tissue engaging member at a select location and to engage the expandable member with a vessel wall, deflating the expandable member, and withdrawing the medical device from the vasculature. The method and medical device can include any number of the features described above.

While the disclosed subject matter is described herein in terms of certain preferred embodiments, those skilled in the art will recognize that various modifications and improvements can be made to the disclosed subject matter without departing from the scope thereof. Moreover, although individual features of one embodiment of the disclosed subject matter can be discussed herein or shown in the drawings of the one embodiment and not in other embodiments, it should be apparent that individual features of one embodiment can be combined with one or more features of another embodiment or features from a plurality of embodiments.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

1. A medical device comprising:
a tubular member having a proximal end and distal end defining a longitudinal axis therebetween;
an expandable member proximate the distal end of the tubular member, the expandable member having a length and_at least one axial fold along its length_in a deflated condition;
a tissue engaging member disposed over an exterior surface of the expandable member, the tissue engaging member extending from of the tubular member and_comprising at least one straight wire extending axially along the length of the expandable member;
a therapeutic agent disposed on at least the expandable member or the tissue engaging member; and
wherein the at least one straight wire of the tissue engaging member is located within the at least one fold of the expandable member when in the deflated condition, and wherein the tissue engaging member is configured for deployment at a select location upon inflation of the expandable member with the at least one straight wire extending substantially axially along the length of the expandable member when inflated.

2. The medical device of claim 1, wherein the expandable member includes a plurality of folds extending substantially axially along the length of the expandable member_in the deflated condition with at least one straight wire located within each fold.

3. The medical device of claim 2, wherein one straight wire of the tissue engaging member is located in substantially the middle of each fold of the expandable member when in the deflated condition.

4. The medical device of claim 1, wherein a distal end of the at least one straight wire of the tissue engaging member is fixedly attached proximate to a distal end of the expandable member.

5. The medical device of claim 4, wherein a proximal end of the at least one straight wire of the tissue engaging member is attached to a slidable collar disposed on the tubular member proximate the proximal end of the expandable member.

6. The medical device of claim 5, wherein the collar is made of a polymeric extrusion having at least one sub-lumen in the wall of the extrusion.

7. The medical device of claim 6, wherein the proximal end of the at least one straight wire of the tissue engaging member is secured in a corresponding sub-lumen of the collar.

8. The medical device of claim 4, wherein the tissue engaging member further comprises a non-linear portion at the proximal end of the least one straight wire, the non-linear portion having a proximal end fixedly attached to the tubular member proximate the proximal end of the expandable member.

9. The medical device of claim 4, wherein the tissue engaging member further comprises a non-linear portion adjacent to the straight wire of the tissue engaging member and located inside a fold of the expanded member when in the deflated condition, and the proximal end of the at least one straight wire of the tissue engaging member is fixedly attached proximate to a proximal end of the expandable member.

10. The medical device of claim 1, wherein the tissue engaging member further comprises a non-linear portion adjacent to the straight wire of the tissue engaging member and located inside a fold of the expanded member when in the deflated condition.

11. The medical device of claim 1, wherein the tissue engaging member further comprises a 2-dimensional array portion adjacent to the straight wire of the tissue engaging member and located inside a fold of the expanded member when in the deflated condition.

12. The medical device of claim 1, wherein at least a portion of the at least one straight wire of the tissue engaging member is secured to a surface of the expandable member.

13. The medical device of claim 1, wherein the at least one straight wire of the tissue engaging member is nitinol, stainless steel, elgiloy, or cobalt-chromium alloy.

14. The medical device of claim 1, wherein the expandable member is cut from a single nitinol tube.

15. The medical device of claim 1, wherein the therapeutic agent includes an excipient, plasticizer, or surfactant, or combinations thereof.

16. The medical device of claim 1, wherein the tissue engaging member has a tissue engaging member to artery ratio in an expanded state is between about 1% and about 50%.

17. The medical device of claim 1, wherein the tissue engaging member has a tissue engaging member to artery ratio in an expanded state is between about 2.5% and about 25%.

18. The medical device of claim 1, wherein the at least one straight wire of the tissue engaging member has a diameter or width between about 0.05 microns to about 250 microns.

19. A method of delivering a therapeutic agent comprising:
delivering at least a portion of a medical device within a vasculature, the medical device including:
a tubular member having a proximal end and distal end defining a longitudinal axis therebetween,
an expandable member proximate the distal end of the tubular member, the expandable member having a length and at least one axial fold along its length in a deflated condition,
a tissue engaging member disposed over an exterior surface of the expandable member, the tissue engaging member extending from the tubular member and comprising at least one straight wire extending axially along the length of the expandable member, the at least one straight wire located within the at least one axial fold of the expanded member, and
a therapeutic agent disposed on at least the expandable member or the tissue engaging member;
inflating the expandable member to deploy the tissue engaging member at a select location and to engage the therapeutic agent with a vessel wall and wherein the at least one straight wire extends substantially axially along the length of the expandable member when inflated;
deflating the expandable member; and
withdrawing the medical device from the vasculature.

20. The method of claim 19, wherein the expandable member includes a plurality of folds in a deflated condition with at least one straight wire located within each fold.

21. The method of claim 20, wherein one straight wire of the tissue engaging member is located in substantially the middle of each fold of the expandable member when in the deflated condition.

22. The method of claim 19, wherein a distal end of the at least one straight wire of the tissue engaging member is fixedly attached proximate to a distal end of the expandable member.

23. The method of claim 22, wherein a proximal end of the at least one straight wire of the tissue engaging member is attached to a slidable collar disposed on the tubular member proximate the proximal end of the expandable member.

24. The method of claim 22, wherein the tissue engaging member further comprises a non-linear portion at the proximal end of the least one straight wire, the non-linear portion having a proximal end fixedly attached to the tubular member proximate the proximal end of the expandable member.

25. The method of claim 19, wherein the expanded member is under negative pressure before inflation.

26. A medical device comprising:
a tubular member having a proximal end and distal end defining a longitudinal axis therebetween;
an expandable member proximate the distal end of the tubular member, the expandable member having a length and at least one axial fold along its length in a deflated condition;
a tissue engaging member disposed over an exterior surface of the expandable member, the tissue engaging member extending from the distal end of the tubular member and_comprising at least one straight wire extending axially along the length_of the expandable member; and
wherein the at least one straight wire of the tissue engaging member is located within the at least one fold of the expanded member when in the deflated condition, and wherein the tissue engaging member is configured for deployment at a select location upon inflation of the expandable member with the at least one straight wire extending substantially axially along the length of the expandable member when inflated.

27. A method of treating a vasculature comprising:
delivering at least a portion of a medical device within a vasculature, the medical device including:
a tubular member having a proximal end and distal end defining a longitudinal axis therebetween,
an expandable member proximate the distal end of the tubular member, the expandable member having a length and at least one axial fold, and
a tissue engaging member disposed along an exterior surface of the expandable member, the tissue engaging member extending from the distal end of the tubular member and comprising at least one straight wire extending axially along the length_of the expandable member, the at least one straight wire located within the at least one axial fold of the expandable member;
inflating the expandable member to deploy the tissue engaging member at a select location and to engage the expandable member with a vessel wall with the at least one straight wire extending substantially axially along the length of the expandable member when inflated;
deflating the expandable member; and
withdrawing the medical device from the vasculature.
